(19) United States
(12) Patent Application Publication
(10) Pub. No.: US 2026/0294865 A1
(43) Pub. Date: Oct. 1, 2026

(54) AUTOPHAGY ACTIVATOR AND AUTOPHAGY ACTIVATION COMPOSITION

(71) Applicant: MARUZEN PHARMACEUTICALS CO., LTD., Hiroshima (JP)

(72) Inventor: Susumu YOSHINO, Hiroshima (JP)

(21) Appl. No.: 19/480,218

(22) PCT Filed: Apr. 4, 2024
(86) PCT No.: PCT/JP2024/013935

§ 371 Date: Oct. 30, 2025

(30) Foreign Application Priority Data
May 10, 2023 (JP) 2023-077829

(51) Int. Cl. A61K 31/352; A61K 9/00; A61K 9/08; A61K 9/20; A61K 9/48; A61K 47/02; A61K 47/12; A61K 47/40; A61K 47/46

(52) U.S. Cl. CPC A61K 31/352; A61K 9/0095; A61K 9/08; A61K 9/2018; A61K 9/2068; A61K 9/4825; A61K 9/4858; A61K 9/4866; A61K 47/02; A61K 47/12; A61K 47/40; A61K 47/46

(57) ABSTRACT

An autophagy activator includes a compound represented by the structural formula (1), a compound represented by the structural formula (2), or both.

### TECHNICAL FIELD

The present invention relates to autophagy activators and autophagy activation compositions.

### BACKGROUND OF THE INVENTION

Autophagy is one mechanism for degrading unnecessary substances within cells. It has been known that autophagy enhances cellular metabolisms, and contributes to cellular health. In addition, it is considered that maintenance of autophagy activities is associated with prevention of aging or longevity. Thus, research has been conducted to discover the specific mechanism of autophagy.

The elderly population ratio (proportion of population over 65 years old) has been increasing year by year in Japan, and it is important to improve quality of life and health spans in the aging society. Therefore, the activation of autophagy has attracted attention.

There is hitherto proposed an autophagy activator including, as an active ingredient, at least one selected from the group consisting of heparinoid, glycyrrhizic acid and a salt of glycyrrhizic acid, tocopherol and a derivative of tocopherol, ε-aminocaproic acid and a salt of ε-aminocaproic acid, nicotinic acid amide, urea, panthenol, and vitamin A (see, for example, Patent Document 1).

However, there is still a strong demand for novel materials that exhibit an excellent autophagy activation effect, are highly safe, and can be widely used as substances for drink or food products, pharmaceutical products, quasi-drugs, research reagents, and the like. Rapid development of such materials is currently desired.

### RELATED-ART DOCUMENT

### Patent Document

Patent Document 1: Japanese Patent Application Laid-Open No. 2021-172641

### SUMMARY OF THE INVENTION

### Problems to be Solved by the Invention

The present invention aims to solve the above-described various problems existing in the related art, and to achieve the following object. Specifically, the object of the present invention is to provide an autophagy activator and an autophagy activation composition, which have an excellent autophagy activation effect and are highly safe.

### Means for Solving the Problems

As a result of intensive studies conducted by the present inventors to solve the above-described problems, the present inventors have found that a compound represented by the following structural formula (1) and a compound represented by the following structural formula (2) have an excellent autophagy activation effect and are highly safe, and therefore are effective materials for activation of autophagy.

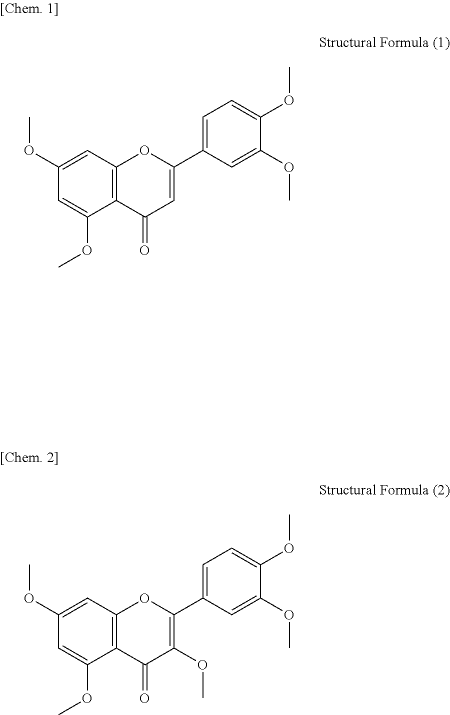 [SMILES: COc1cc(OC)c2c(=O)cc(-c3ccc(OC)c(OC)c3)oc2c1 ; COc1cc(OC)c2c(=O)c(OC)c(-c3ccc(OC)c(OC)c3)oc2c1]

The present invention is based on the insights of the present inventors, and the means for solving the above problems are as follows.

<1> An autophagy activator includes a compound represented by the following structural formula (1), a compound represented by the following structural formula (2), or both.

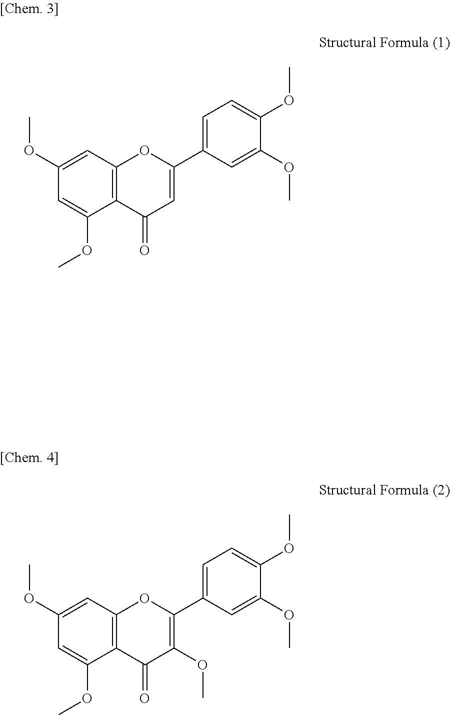 [SMILES: COc1cc(OC)c2c(=O)cc(-c3ccc(OC)c(OC)c3)oc2c1 ; COc1cc(OC)c2c(=O)c(OC)c(-c3ccc(OC)c(OC)c3)oc2c1]

<2> An autophagy activation composition includes the autophagy activator according to <1>.

### Effects of the Invention

The autophagy activator and autophagy activation composition of the present invention can solve the above-described various problems existing in the related art, can achieve the above object, and can provide an autophagy activator and an autophagy activation composition, which have an excellent autophagy activation effect and are highly safe.

### DETAILED DESCRIPTION OF THE INVENTION

### (Autophagy Activator)

The autophagy activator of the present invention includes, as an active ingredient, a compound represented by the following structural formula (1), a compound represented by the following structural formula (2), or both, and may further include other substances, as necessary.

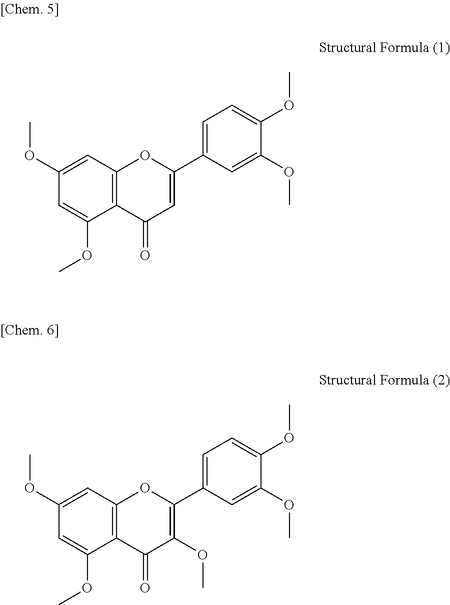 [SMILES: COc1cc(OC)c2c(=O)cc(-c3ccc(OC)c(OC)c3)oc2c1 ; COc1cc(OC)c2c(=O)c(OC)c(-c3ccc(OC)c(OC)c3)oc2c1]

In the present specification, the “activation of autophagy” refers to functions of autophagy being enhanced compared to when the autophagy activator is not used. The level of the activation of autophagy is not particularly limited, and may be appropriately selected according to the intended purpose.

### Compound Represented by Structural Formula (1)

The English name of the compound represented by the following structural formula (1) is 2-(3,4-dimethoxyphenyl)-5, 7-dimethoxy-4H-chromen-4-one

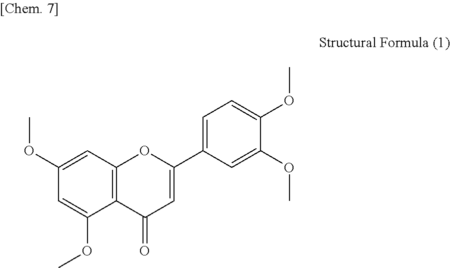 [SMILES: COc1cc(OC)c2c(=O)cc(-c3ccc(OC)c(OC)c3)oc2c1]

### <Compound Represented by Structural Formula (2)>

The English name of the compound represented by the following structural formula (2) is 3, 7-dimethoxy-2-(3, 4, 5-trimethoxyphenyl)-4H-chromen-4-one

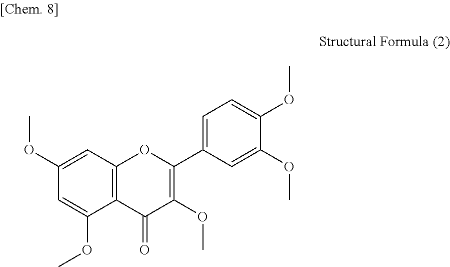 [SMILES: COc1cc(OC)c2c(=O)c(OC)c(-c3ccc(OC)c(OC)c3)oc2c1]

It has not been known at all that the compound represented by the structural formula (1) and the compound represented by the structural formula (2) have an excellent autophagy activation effect and are effective materials for autophagy activation. This is a novel insight of the present inventors.

The compound represented by the structural formula (1) and the compound represented by the structural formula (2) are known compounds, and commercially available products can be used as the above compounds.

Since the compound represented by the structural formula (1) and the compound represented by the structural formula (2) have an excellent autophagy activation effect, the above compounds can be used as active ingredients of an autophagy activator.

The autophagy activator may be composed of the compound represented by the structural formula (1), the compound represented by the structural formula (2), or both, or may be formulated as a preparation including the compound represented by the structural formula (1), the compound represented by the structural formula (2), or both.

Either or both the compound represented by the structural formula (1) and the compound represented by the structural formula (2) may be used. In the case where both the compound represented by the structural formula (1) and the compound represented by the structural formula (2) are used, a mass ratio between the compound represented by the structural formula (1) and the compound represented by the structural formula (2) is not particularly limited, and may be appropriately selected according to the intended purpose.

The compound represented by the structural formula (1), the compound represented by the structural formula (2), or both can be formulated into any dosage form, such as a powder, granules, tablets, or a liquid according to a common practice using a pharmaceutically acceptable carrier, such as dextrin, cyclodextrin, or the like, and any other auxiliary agents. For the formulation, for example, excipients, binders, disintegrants, lubricants, stabilizers, flavoring agents or masking agents, and the like can be used as the auxiliary agents.

The autophagy activator can be used by being blended in another composition (e.g., a below-described autophagy activation composition, etc.), and also can be used as an oral administration agent, such as tablets, a powder, capsules, granules, an extract, or a syrup; a parenteral administration agent, such as an injection, a drip infusion, or a suppository; an ointment, an eye drop, an external liquid, a patch, or the like.

An amount of the compound represented by the structural formula (1), the compound represented by the structural formula (2), or both in the formulated autophagy activator is not particularly limited, and may be appropriately selected according to the intended purpose.

### <Other Substances>

Other substances are not particularly limited as long as the other substances do not adversely affect the effects of the present invention, and may be appropriately selected according to the usage form of the autophagy activator. Examples of the other substances include the above-described substances that can be used when the autophagy activator is formulated into a preparation. As the other substances, one substance may be used alone, or two or more substances may be used in combination.

An amount of the other substances in the autophagy activator is not particularly limited, and may be appropriately selected according to the intended purpose.

Usage of the autophagy activator is not particularly limited, and may be appropriately selected according to the intended purpose. Examples of the usage include oral use, parenteral use, external use, and the like. The usage is preferably oral use.

A dosage form of the autophagy activator is not particularly limited, and may be appropriately selected from dosage forms known in the related art according to the intended purpose.

A production method for the autophagy activator in a desired dosage form is not particularly limited, and may be appropriately selected from methods known in the related art.

An administration dose, administration site, administration period, administration interval, and the like of the autophagy activator are not particularly limited, and may be appropriately selected according to the intended purpose.

The autophagy activator can activate autophagy through the autophagy activation effect of the compound represented by the structural formula (1), the compound represented by the structural formula (2), or both. This activation of autophagy can enhance cellular metabolisms, can improve cellular health, can increase activities of autophagy, which are reduced by aging, or can prevent reduction in activities of autophagy, which are caused by aging, can slow aging, and can achieve longevity. In addition to the use above, the autophagy activator can be used in all applications for which exhibition of the autophagy activation effect is beneficial.

Since the autophagy activator has an excellent autophagy activation effect and is highly safe, the autophagy activator can be used in various uses, such as pharmaceutical products, quasi-drugs, oral compositions, cosmetics, reagents for studies on the mechanism of actions of autophagy activation, and the like. For example, the autophagy activator can be suitably used as an active ingredient of the below-described autophagy activation composition. In this case, the compound represented by the structural formula (1), the compound represented by the structural formula (2), or both may be blended as they are, or the compound represented by the structural formula (1), the compound represented by the structural formula (2), or both may be formulated, and the formulated preparation may be blended in the autophagy activation composition.

The autophagy activator may also include, as an active ingredient, another substance exhibiting an autophagy activation effect that is blended together with the compound represented by the structural formula (1), the compound represented by the structural formula (2), or both, as necessary.

The autophagy activator of the present invention is suitably applied to humans, but can also be applied to animals other than humans (e.g., mice, rats, hamsters, dogs, cats, cattle, pigs, monkeys, etc.) as long as the functions and effects of the autophagy activator can be exhibited.

### (Autophagy Activation Composition)

The autophagy activation composition of the present invention includes the autophagy activator of the present invention, and may further include other substances, as necessary.

### <Autophagy Activator>

The autophagy activator is the above-described autophagy activator of the present invention.

An amount of the autophagy activator in the autophagy activation composition is not particularly limited, and may be appropriately adjusted according to the form of the autophagy activation composition or the like. The amount of the autophagy activator, which is converted as the amount of the compound represented by the structural formula (1), the compound represented by the structural formula (2), or both, is preferably 0.0001 percent by mass to 20 percent by mass, and more preferably 0.0001 percent by mass to 10 percent by mass. The autophagy activation composition may be composed of the autophagy activator.

### <Other Substances>

Other substances in the autophagy activation composition are not particularly limited, and may be appropriately selected according to the usage form of the autophagy activation composition. Examples of the other substances include the same substances as the other substances described in the section of the autophagy activator. As the other substances, one substance may be used alone, or two or more substances may be used in combination.

An amount of the other substances in the autophagy activation composition is not particularly limited, and may be appropriately selected according to the intended purpose.

### <Form>

A form of the autophagy activation composition is not particularly limited, and may be appropriately selected according to the intended purpose. Examples of the form include pharmaceutical products, quasi-drugs, food or drink products, and the like.

The autophagy activation composition can be used regularly, and can very effectively exhibits various physiological effects including an autophagy activation effect through the actions of the compound represented by the structural formula (1), the compound represented by the structural formula (2), or both serving as an active ingredient.

The autophagy activation composition of the present invention is suitably applied to humans, but can also be applied to animals other than humans (e.g., mice, rats, hamsters, dogs, cats, cattle, pigs, monkeys, etc.) as long as the functions and effects of the autophagy activation composition can be exhibited.

Usage of the autophagy activation composition is not particularly limited, and may be appropriately selected according to the intended purpose. Examples of the usage include oral use, parenteral use, external use, and the like. The usage is preferably oral use.

The oral composition is not particularly limited, and may be appropriately selected according to the intended purpose. Examples of the oral composition include oral administration preparations, food or drink products, and the like. The food or drink products refer to products that pose little risk of harming human health and can be consumed in ordinary social life through oral administration or gastrointestinal administration, and are not limited to the categories according to the administrative classification, such as food products, pharmaceutical products, quasi-drugs, and the like. Accordingly, the food or drink products encompass a wide range of products, such as general food products, health food products (functional food or drink products), health-promoting food products (foods for specified health uses, foods with nutrient function claims, foods with function claims), quasi-drugs, pharmaceutical products, and the like, which are for oral intake.

A type of the oral composition is not particularly limited, and may be appropriately selected according to the intended purpose. Examples of the type of the oral composition include: beverages, such as tea beverages, soft drinks, carbonated beverages, nutritional beverages, fruit-based beverages, lactic beverages, alcoholic beverages, coffee beverages, coffee-containing soft drinks, and the like (including concentrates of the foregoing beverages and powders for preparing the foregoing beverages); frozen desserts such as ice cream, sherbet, shaved ice, and the like; noodles, such as buckwheat noodles, udon noodles, starch noodles, Gyoza skins, Shumai skins, Chinese noodles, instant noodles, and the like; confectionary, such as hard candies, candies, chewing gums, chocolate, tablets, snacks, biscuits, jelly, jams, creams, sweet pastries, bread, and the like; fishery products, such as crabs, salmon, clams, tuna, sardine, shrimp, bonito, mackerel, whale, oysters, Pacific saury, squid, Anadara broughtonii, scallops, abalone, sea urchin, salmon roe, Sulculus diversicolor supertexta, and the like; fish or meat processed foods, such as fish paste products, ham, sausages, and the like; dairy products, such as processed milk, fermented milk, and the like; fats and oils, or fat and oil processed food products, such as salad oil, tempura oil, margarines, mayonnaise, shortening, whipping creams, dressing, and the like; seasonings, such as sauces and dipping sauces; retort pouch foods, such as curry, stew, Oyakodon, gruel, rice soups, Chukadon, a bowl of rice with breaded pork cutlets, a bowl of rice with tempura, Unadon, Hayashi rice, Oden, Mapo tofu, a bowl of rice with beef, Bolognese sauces, egg soup, omelet with rice, Gyoza, Shumai, hamburgers, meat balls, and the like; deli dishes, such as salads, pickles, and the like; various forms of health, cosmetic, or nutritional supplementary foods; pharmaceutical products and quasi-drugs, such as tablets, powders, capsules, granules, extracts, syrups, drink preparations, lozenges, mouthwash, and the like; oral fresheners used inside the mouth, such as mouth freshener and breath freshener; toothpaste; and the like.

A production method for the autophagy activation composition is not particularly limited, and may be appropriately selected according to the form of the autophagy activation composition or the like.

A usage amount, usage period, usage interval, and the like of the autophagy activation composition are not particularly limited, and may be appropriately selected according to the intended purpose.

As described above, the autophagy activator and autophagy activation composition of the present invention have an excellent autophagy activation effect.

Accordingly, the present invention also relates to an autophagy activation method of administering the autophagy activator, the autophagy activation composition, or both to an individual.

### EXAMPLES

Test examples and formulation examples of the present invention will be described hereinafter, but the present invention is not limited to the following test examples and formulation examples in any way.

### Test Example 1: Evaluation of Autophagy Activities by tfLC3 Test

An autophagy activation effect was examined by a tfLC3 test using the compound represented by the structural formula (1) (manufactured by INDOFINE Chemical Company) and the compound represented by the structural formula (2) (manufactured by INDOFINE Chemical Company) as test samples.

The tfLC3 test is a test performed by using tfLC3, in which LC3 that is an autophagosome marker protein is tandemly tagged with fluorescent proteins mRFP (RFP: Red Fluorescent Protein) and EGFP (GFP: Green Fluorescent Protein) (Autophagy 2007; 3:452-460. (PubMed: 17534139) DOI: 10.4161/auto. 4451).

In a cell undergoing autophagy, LC3 dots increase. However, LC3 is accumulated in a cell even if degradation due to autophagy is inhibited. Therefore, an increase of the LC3 dots alone cannot be used to conclude that autophagy is enhanced. Therefore, enhancement and inhibition of autophagy can be distinguished by examining the maturation process of autophagosomes (process of fusion of autophagosomes with lysosomes, or degradation in autolysosomes after fusion).

A fluorescent signal (green) of GFP of the tfLC3 is attenuated in the environment within the lysosomes, but RFP (red) is stable in the same environment. By using this characteristic of tfLC3 that fluorescence becomes red due to quenching of GFP in the process of maturing autophagosomes into autolysosomes, a GFP/RFP ratio within a cell is analyzed in the tfLC3 test to determine whether or not a test sample added to a medium has an autophagy activation capability.

The specific experimental method is as follows.

HeLa cells stably expressing tfLC3 were seeded in a 96-well plate (Perkin Elmer #6055302 Cell Carrier 96 Ultra Black 96 well, Clear Bottom, with Lid), and the HeLA cells were cultured in a growth medium [Growth medium (DMEM (Sigma D6546) supplemented with 10% FBS and 4 mM L-glutamine] in a 5% CO₂ incubator for 24 hours. Then, the medium was replaced with a medium to which a test sample was added (see Table 1 for the final concentration of the test sample), followed by culturing for 24 hours.

As a negative control, cells cultured in a medium including 0.1% dimethyl sulfoxide (DMSO) were used.

The GFP/RFP value (may be referred to as a “ratio with respect to DMSO” hereinafter) measured in the cells cultured in the test sample-added medium was calculated by determining the GFP/RFP value measured in the negative control as 1. The results are presented in Table 1.

A test sample that has a lower value for the GFP/RFP ratio than the value for the GFP/RFP ratio of the negative control can be determined as having an autophagy activation effect.

TABLE 1

| | Final | Ratio with | CV value |
|---|---|---|---|
| Test sample | concentration | respect to DMSO | (%) |
| Compound | 10 μM | 0.847 | 0.061 |
| represented | | | |
| by structural | | | |
| formula (1) | | | |
| Compound | 10 μM | 0.848 | 0.059 |
| represented by | | | |
| structural | | | |
| formula (2) | | | |

It was confirmed from the results above that the compound represented by the structural formula (1) and the compound represented by the structural formula (2) had the autophagy activation effect.

### Formulation Example 1

A tablet having the following composition was produced according to a common practice.
Compound represented by structural formula (1): 50.0 mg Grape extract: 100.0 mg
β-nicotinamide mononucleotide: 5.0 mg Dolomite: 23.4 mg (including 20% calcium and 10% magnesium)
Vitamin C: 13.4 mg
Maltitol: 96.2 mg
Sucrose fatty acid ester: 12.0 mg

### Formulation Example 2

An oral liquid preparation having the following composition was produced according to a common practice.

<Composition in one ampoule (100 mL per ampoule)>
Compound represented by the structural formula (2): 0.3 percent by mass
Sorbitol: 12.0 percent by mass
Sodium benzoate: 0.1 percent by mass
Flavoring agent: 1.0 percent by mass
Calcium sulfate: 0.5 percent by mass
Purified water: remainder

### Formulation Example 3

A coffee beverage having the following composition was produced according to a common practice.

Compound represented by the structural formula (1): 0.1 percent by mass
Coffee extract: 40 percent by mass (L (brightness)=20, Brix scale=3)
Maltitol: 2 percent by mass
Flavoring agent: appropriate amount
Water: remainder

### Formulation Example 4

Capsules having the following composition were produced according to a common practice. As capsule shells, hard gelatin No. 1 capsules were used.

<Composition in 1 capsule (200 mg per capsule)>
Compound represented by the structural formula (1): 15.0 mg
Compound represented by the structural formula (2): 15.0 mg
Corn starch: 70.0 mg
Lactose: 80.0 mg
Calcium lactate: 10.0 mg
Hydroxypropyl cellulose (HPC-L): 10.0 mg

This application claims priority under Japanese Patent Application No. 2023-077829 filed May 10, 2023, and the entire contents of Japanese Patent Application No. 2023-077829 are incorporated herein by reference.

1. An autophagy activator, comprising:
a compound represented by the following structural formula (1), a compound represented by the following structural formula (2), or both

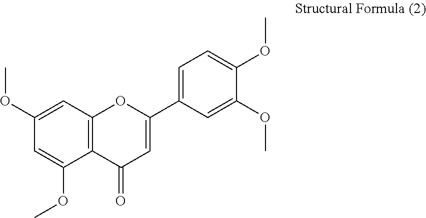 [SMILES: COc1cc(OC)c2c(=O)cc(-c3ccc(OC)c(OC)c3)oc2c1]

2. An autophagy activation composition, comprising:
the autophagy activator of claim 1; and
at least one selected from the group consisting of dextrin, cyclodextrin, an excipient, a binder, a disintegrant, a lubricant, a stabilizer, a flavoring agent, and a masking agent.

3. A method of activating autophagy, comprising:
administering the autophagy activator of claim 1 to an individual to activate autophagy.
